(19) United States
(12) Patent Application Publication
(10) Pub. No.: US 2024/0307430 A1
(43) Pub. Date: Sep. 19, 2024

(54) PHARMACEUTICAL COMPOSITION CONTAINING NICOTINAMIDE ADENINE DINUCLEOTIDE (NAD) AND CD38 INHIBITOR AND APPLICATION THEREOF

(71) Applicant: HEFEI KNATURE BIO-PHARM CO., LTD., Hefei (CN)

(72) Inventors: Junbo GE, Hefei (CN); Aijun SUN, Hefei (CN); Wei LUO, Hefei (CN); Wei WANG, Hefei (CN); Kanglin WANG, Hefei (CN); Lei WANG, Hefei (CN); Beijian ZHANG, Hefei (CN); Zheng DONG, Hefei (CN); Xinyu WENG, Hefei (CN); Yongchao ZHAO, Hefei (CN)

(21) Appl. No.: 18/671,968

(22) Filed: May 22, 2024

Related U.S. Application Data
(63) Continuation of application No. PCT/CN2022/130472, filed on Nov. 8, 2022.

(30) Foreign Application Priority Data
Nov. 30, 2021 (CN) 202111448300.2

(51) Int. Cl. A61K 31/7084; A61K 31/4709; A61P 9/00

(52) U.S. Cl. CPC A61K 31/7084; A61K 31/4709; A61P 9/00

(57) ABSTRACT

A pharmaceutical composition, including nicotinamide adenine dinucleotide (NAD) and a CD38 inhibitor in a weight ratio of 1-5:5-1, where the CD38 inhibitor is compound 78c. A pharmaceutical preparation including the pharmaceutical composition and an application of the pharmaceutical composition in the prevention and/or treatment of doxorubicin-induced cardiotoxicity are further provided.

### CROSS-REFERENCE TO RELATED APPLICATIONS

This application is a continuation of International Patent Application No. PCT/CN2022/130472, filed on Nov. 8, 2022, which claims the benefit of priority from Chinese Patent Application No. 202111448300.2, filed on Nov. 30, 2021. The content of the aforementioned application, including any intervening amendments thereto, is incorporated herein by reference in its entirety.

### TECHNICAL FIELD

This application relates to pharmaceutical compositions, and more particularly to a pharmaceutical composition containing nicotinamide adenine dinucleotide (NAD) and a CD38 inhibitor, and an application thereof.

### BACKGROUND

Anthracyclines, represented by doxorubicin, are a class of important chemotherapeutic drugs that have been widely used in the treatment of solid and hematological malignancies, such as breast cancer, acute leukemia, and lymphoma. However, the doxorubicin-induced cardiotoxicity (DIC) has severely limited its clinical application. Although the emerging liposomal doxorubicin has an improved tumor-targeting activity, the cardiotoxicity caused thereby still cannot be completely eliminated. The mortality of patients with doxorubicin-induced congestive heart-failure (CHF) reaches 50%. Currently, there is no effective therapy for DIC, and dexrazoxane is the only drug approved by the FDA for the prevention of DIC. However, considering its poor efficacy and significant side effects, it is urgently needed to develop an effective drug for the prevention and treatment of DIC.

Main mechanisms of DIC include mitochondrial damage, nuclear DNA damage, and production of a large amount of reactive oxygen. Nicotinamide adenine dinucleotide (NAD) is an important coenzyme of many dehydrogenases involved in the cellular metabolism, and participates in the mitochondrial electron transport chain, and plays a key role in maintaining mitochondrial homeostasis and repairing the cellular nuclear damage.

Numerous preclinical studies have shown that the decline in the intracellular NAD level would accelerate the aging and progression of various diseases, such as neurodegenerative diseases, diabetes mellitus, ischemic heart disease (IHD), and heart failure. Exogenous supplementation or stimulation of endogenous NAD synthesis can effectively slow down the aging and prevent various diseases. Recent studies have found that the loss of NAD homeostasis would contribute to the development of DIC, and increasing the cardiac NAD level can reduce the occurrence of DIC. Therefore, NAD is expected to be an important target for the prevention and treatment of DIC and other related diseases. Although extensive animal experiments have demonstrated the feasibility of exogenous NAD supplementation to increase the NAD level in tissues, similar results have not been observed in the clinical trials. The poor NAD supplementation efficiency has greatly limited the application of NAD in clinical disease prevention and treatment.

### SUMMARY

An object of this application provides a pharmaceutical composition containing nicotinamide adenine dinucleotide (NAD) and a CD38 inhibitor to reduce cardiotoxicity induced by cancer chemotherapy drugs.

Technical solutions of this application are described as follows.

This application provides a pharmaceutical composition comprising a nicotinamide adenine dinucleotide (NAD) and a CD38 inhibitor, wherein the NAD can be dosed in the oxidized or reduced state.

In an embodiment, the CD38 inhibitor is compound 78c which is also known as CD38-IN-78c or MDK-7553 with a CAS number of 1700637-55-3.

In an embodiment, a weight ratio of the NAD to CD38 inhibitor in the pharmaceutical composition is 1-20:20-1.

In an embodiment, the weight ratio of the NAD to CD38 inhibitor in the pharmaceutical composition is 1-10:10-1.

In an embodiment, the weight ratio of the NAD to CD38 inhibitor in the pharmaceutical composition is 1-5:5-1.

This application further provides a pharmaceutic preparation comprising the pharmaceutical composition described above.

In an embodiment, the pharmaceutic preparation is in a liquid dosage form, a solid dosage form, a semi-solid dosage form or a gaseous dosage form.

In an embodiment, the liquid dosage form is selected from the group consisting of solution, injection, infusion solution, and oral liquid; the solid dosage form is selected from the group consisting of tablet, capsule, powder, and granule; the semi-solid dosage form is selected from the group consisting of ointment and gel; and the gaseous dosage form is selected from the group consisting of aerosol and spray.

This application further provides a method for preventing and/or treating cardiotoxicity induced by an anti-tumor drug in a subject in need thereof, comprising administering a therapeutically effective amount of the above pharmaceutical composition or the above pharmaceutical preparation to the subject.

In an embodiment, the anti-tumor drug is doxorubicin.

This application has the following beneficial effects.

The application provides a nicotinamide adenine dinucleotide (NAD)-containing pharmaceutical composition, which can enhance the efficacy of NAD in the prevention and/or treatment of cardiotoxicity induced by cancer chemotherapeutic agents and expand the therapeutic scope of NAD.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to illustrate the technical solutions in the embodiments of the present disclosure or in the prior art more clearly, the drawings required in the description of the embodiments or the prior art will be briefly described below. Obviously, presented in the drawings are merely some embodiments of the present disclosure, which are not intended to limit the disclosure. For those skilled in the art, other drawings may also be obtained according to the drawings provided herein without paying creative efforts.

FIG. 1 shows CD38 expression in endothelial cells after doxorubicin treatment;

FIG. 2 shows comparison of mice in different groups in terms of the NAD″ content in cardiac tissues; and

FIGS. 3a and 3b show comparison of mice in different groups in terms of cardiac function; where 3a, ejection fraction (EF); and 3b, fractional shortening (FS).

Note: *p<0.05, **p<0.01, and ***p<0.001 in the figures.

### DETAILED DESCRIPTION OF EMBODIMENTS

The present disclosure will be further described in detail below in conjunction with the accompanying drawings and embodiments to make the above objects, features and advantages of the present disclosure clearer. Described above are merely preferred embodiments of the disclosure, which are not intended to limit the disclosure. For those skilled in the art, other embodiments obtained based on these embodiments without paying creative efforts should fall within the scope of the disclosure.

The mice used in the embodiments were purchased from Changzhou Cavens Laboratory Animal Centre. Nicotinamide adenine dinucleotide (NAD) and compound 78c were obtained from Kaifeng Kangnuo Pharmaceutical Co. and Selleck Chemicals, respectively. Doxorubicin was obtained from American Sigma company. NAD detection kits were purchased from Biyuntian Biological Co., Ltd. The animal experiments were approved by Ethics Committee of Zhongshan Hospital, Fudan University.

Model of Doxorubicin-induced cardiotoxicity (DIC) can be found in the literature: “Doxorubicin Blocks Cardiomyocyte Autophagic Flux by Inhibiting Lysosome Acidification. Circulation. 2016; 133:1668-1687”.

The inhibitory activity of the compound 78c against CD38 has been demonstrated in the literature “A Potent and Specific CD38 Inhibitor Ameliorates Age-Related Metabolic Dysfunction by Reversing Tissue NAD+ Decline, cell Metabolism. 2018.03.016”.

### Example 1 Construction of DIC Model

8-week-old C57BL/6 male mice were injected with 5 mg/kg of doxorubicin (DOX) or normal saline via the tail vein, and the administration was performed once a week for four consecutive weeks. Four weeks after the last administration, the mice were subjected to echocardiography to detect the cardiac function, and myocardial tissues of the mice were taken to detect the NAD⁺ content.

### Example 2

### 1. Blank Control Group

Mice were intraperitoneally injected with the same volume of saline as that of the experimental group once a day, and the duration cycle was the same as that of the DIC model. After administration, mice were subjected to echocardiography to detect the cardiac function, and myocardial tissues were taken from mice to detect NAD⁺ content.

### 2. NAD Synthase Nampt Agonist Group

NAD synthase Nampt agonist P7C3 was dissolved in DMSO. Mice were injected intraperitoneally with 20 mg/kg of NAD synthase Nampt agonist P7C3. P7C3 was administered once a day for the same duration as that of the DIC model. After administration, myocardial tissues of mice were taken to detect NAD⁺ content.

### 3. NAD-Depleting Enzyme PARP Inhibitor Group

NAD-depleting enzyme PARP inhibitor Olaparib was dissolved in DMSO. Mice were injected intraperitoneally with 10 mg/kg of NAD-depleting enzyme PARP inhibitor Olaparib. Olaparib was administered once a day for the same duration as that of the DIC model. After administration, myocardial tissues of mice were taken to detect NAD⁺ content.

### 4. CD38 Inhibitor Group

CD38 inhibitor 78c was dissolved in DMSO. Mice were injected intraperitoneally with 10 mg/kg of CD38 inhibitor 78c. 78c was administered once a day for the same duration as that of the DIC model. After administration, myocardial tissues of mice were taken to detect NAD⁺ content.

### 5. NAD Synthase Nampt Agonist+DOX Group

NAD synthase Nampt agonist P7C3 was dissolved in DMSO. Mice were injected intraperitoneally with 20 mg/kg NAD synthase Nampt agonist P7C3 at 3 hours before doxorubicin injection in the DIC model. P7C3 was administered once a day that lasted until the endpoint of the DIC model. At the end of the model, myocardial tissues of mice were taken to detect NAD⁺ content.

### 6. NAD-Depleting Enzyme PARP Inhibitor+DOX Group

NAD-depleting enzyme PARP inhibitor Olaparib was dissolved in DMSO. Mice were injected intraperitoneally with 10 mg/kg of NAD-depleting enzyme PARP inhibitor Olaparib at 3 hours prior to doxorubicin injections in the DIC model. The Olaparib was administered once a day that lasted until the endpoint of the DIC model. At the end of the model, myocardial tissues of mice were to detect NAD⁺ content.

### 7. CD38 Inhibitor+DOX Group

CD38 inhibitor 78c was dissolved with DMSO. Mice were injected intraperitoneally with 10 mg/kg CD38 inhibitor 78c at 3 hours before doxorubicin injection in the DIC model. 78c was administered once a day until the endpoint of the DIC model. At the end of the model, echocardiography was performed on the mice to detect the cardiac function of the mice, and myocardial tissues were taken from the mice to detect NAD⁺ content.

### 8. NAD+DOX Group

NAD was dissolved in saline. Mice were injected intraperitoneally with 50 mg/kg NAD at 3 hours before doxorubicin injection in the DIC model. NAD was administered once a day, which lasted until the endpoint of the DIC model. At the end of the model, mice were subjected to echocardiography to detect cardiac function, and myocardial tissues were taken from mice to detect NAD⁺ content.

### 9. NAD Synthase Nampt Agonist+NAD+DOX Group

NAD synthase Nampt agonist P7C3 was dissolved in DMSO. Mice were injected intraperitoneally with 50 mg/kg of NAD and 20 mg/kg of NAD synthase Nampt agonist P7C3 at 3 hours before the doxorubicin injection in the DIC model. P7C3 was administered once a day, which lasted until the endpoint of the DIC model. At the end of the model, myocardial tissues were taken from mice to detect NAD″ content.

### 10. NAD-Depleting Enzyme PARP Inhibitor+NAD+DOX Group

NAD-depleting enzyme PARP inhibitor Olaparib was dissolved in DMSO. Mice were injected intraperitoneally with 50 mg/kg NAD and 10 mg/kg NAD-depleting enzyme PARP inhibitor Olaparib at 3 hours prior to the doxorubicin injection in the DIC model. Olaparib was administered once a day, which lasted until the endpoint of the DIC model. At the end of the model, myocardial tissues were taken from mice to detect NAD⁺ content.

### 11. CD38 Inhibitor+NAD+DOX Group

CD38 inhibitor 78c was dissolved in DMSO. Mice were injected intraperitoneally with 50 mg/kg NAD and 10 mg/kg CD38 inhibitor 78c at 3 hours before doxorubicin injection in the DIC model. The CD38 inhibitor 78c was administered once a day, which lasted until the endpoint of the DIC model. At the end of the model, mice were subjected to echocardiography to detect cardiac function, and myocardial tissues were taken from mice to detect NAD⁺ content.

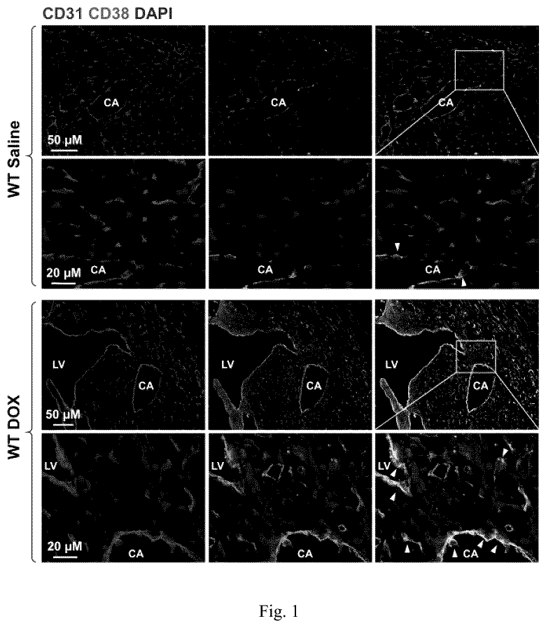

The results showed that CD38 expression was significantly increased in mouse heart tissues after doxorubicin treatment and was mainly showed in the marker CD31 region positive for the endothelial cell (as shown in FIG. 1).

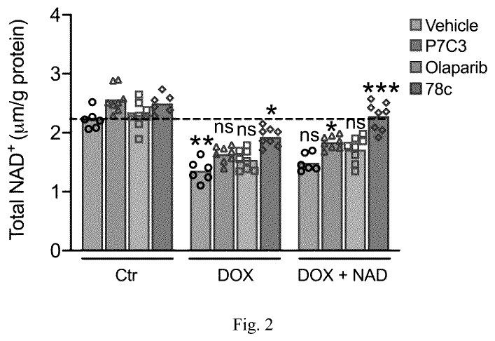

In addition, there are three main NAD depletion pathways in the human body, namely sirtuins signaling pathway, PARP signaling pathway and CD38 pathway. Mice in the embodiments were treated with doxorubicin and simultaneously administered with NAD synthase Nampt agonist P7C3, NAD-depleting enzyme PARP inhibitor Olaparib, NAD-depleting enzyme CD38 inhibitor 78c, and NAD. As shown in FIG. 2, compared with the control group, doxorubicin treatment resulted in a significant decrease in the NAD⁺ content of the cardiac tissues of the mice. NAD or inhibitor 78c alone modestly increased cardiac NAD levels, but the combination of NAD and inhibitor 78c significantly increased cardiac NAD⁺ content. Moreover, the combination of NAD and NAD-depleting enzyme PARP inhibitor Olaparib was not significantly different from the NAD-alone group. The combination of NAD and the Nampt agonist P7C3 was significantly weaker than the combination of NAD and 78c inhibitor in increasing NAD levels.

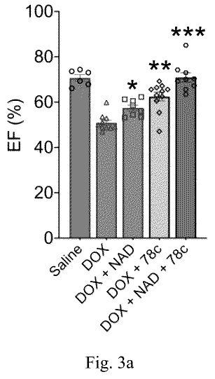

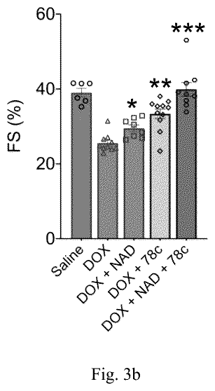

At the end of the model, cardiac functions in mice were evaluated. As shown in FIGS. 3a and 3b, doxorubicin treatment significantly reduced the ejection fraction (EF) and fractional shortening (FS) of the mouse heart; and NAD or 78c alone improved cardiac function, whereas the combination of NAD and 78c significantly improved cardiac function.

It is to be noted that the relational terms such as “first” and “second” are used only to distinguish one entity or operation from another and cannot be understood as one actual relationship or order between these entities or operations. Further, the terms “including”, “comprising”, or any other variant thereof, are intended to cover non-exclusive inclusion, such that a process, method, article, or apparatus comprising a set of elements includes not only listed elements, but also other elements not expressly listed, or other elements that are inherent to such process, method, article, or apparatus. Without further limitation, the fact that an element is defined by the phrase “comprising a . . . ” does not exclude the existence of another same elements in the process, method, article, or apparatus.

Described above are merely preferred embodiments of the disclosure, which are not intended to limit the disclosure. It should be understood that any modifications and replacements made by those skilled in the art without departing from the spirit of the disclosure should fall within the scope of the disclosure defined by the appended claims.

What is claimed is:

1. A pharmaceutical composition, comprising:
nicotinamide adenine dinucleotide (NAD); and
a CD38 inhibitor;
wherein a weight ratio of the NAD to the CD38 inhibitor is 1-5:5-1; and the CD38 inhibitor is compound 78c.

2. A pharmaceutical preparation, comprising:
the pharmaceutical composition of claim 1.

3. The pharmaceutical preparation of claim 2, wherein the pharmaceutical formulation is in a liquid dosage form, a solid dosage form, a semi-solid dosage form, or a gaseous dosage form.

4. The pharmaceutical preparation of claim 3, wherein the liquid dosage form is selected from the group consisting of injection, infusion solution and oral liquid; the solid dosage form is selected from the group consisting of tablet, capsule, powder, and granule; the semi-solid dosage form is selected from the group consisting of ointment and gel; and the gaseous dosage form is selected from the group consisting of aerosol and spray.

5. A method for treating cardiotoxicity induced by an anti-tumor drug in a subject in need thereof, comprising:
administering a therapeutically effective amount of the pharmaceutical composition of claim 1 to the subject.

6. The method of claim 5, wherein the anti-tumor drug is doxorubicin.

7. A method for treating cardiotoxicity induced by an anti-tumor drug in a subject in need thereof, comprising:
administering a therapeutically effective amount of the pharmaceutical preparation of claim 2 to the subject.

8. The method of claim 7, wherein the anti-tumor drug is doxorubicin.
